(19) United States
(12) Patent Application Publication
(10) Pub. No.: US 2013/0280201 A1
(43) Pub. Date: Oct. 24, 2013

(54) FULLY ACYLATED AMINO-FUNCTIONAL ORGANOPOLYSILOXANES

(71) Applicants: Christof Brehm, Burghausen (DE); Gerhard Beer, Burghausen (DE); Markus Merget, Mehring (DE)

(72) Inventors: Christof Brehm, Burghausen (DE); Gerhard Beer, Burghausen (DE); Markus Merget, Mehring (DE)

(73) Assignee: WACKER CHEMIE AG, Munich (DE)

(21) Appl. No.: 13/821,466

(22) PCT Filed: Sep. 19, 2011
(86) PCT No.: PCT/EP11/66201

§ 371 Date: Mar. 7, 2013

(30) Foreign Application Priority Data
Sep. 28, 2010 (DE) 10 2010 041 503.0

(51) Int. Cl. A61K 8/898; A61Q 5/02; A61Q 5/12

(52) U.S. Cl. CPC A61K 8/898; A61Q 5/12; A61Q 5/02
USPC 424/70.122; 556/419; 510/122

(57) ABSTRACT

Fully acylated hydroxyl-terminated organopolysiloxanes bearing pendant aminoalkyl groups are easily prepared by reacting a hydroxyl-terminated, aminoalkyl organopolysiloxane with a carboxylic anhydride in stoichiometric amount relative to amino groups, in the presence of at least one emulsifier.

### CROSS-REFERENCE TO RELATED APPLICATIONS

This application is the U.S. national phase of PCT Appln. No. PCT/EP2011/066201 filed Sep. 19, 2011 which claims priority to German application 10 2010 041 503.0 filed Sep. 28, 2010, the disclosures of which are incorporated in their entirety by reference herein.

### BACKGROUND OF THE INVENTION

1. Field of the Invention

The invention relates to fully acylated amino functional and simultaneously hydroxy-terminated organopolysiloxanes, to aqueous emulsions comprising the organopolysiloxanes, to their use in the cosmetics sector and to the preparation of the organopolysiloxanes.

2. Description of the Related Art

Organopolysiloxanes having amino groups whose amino groups are acylated have already been prior art for some time and are used for example as textile finishing agents. The acylation here serves primarily to effectively reduce thermoyellowing.

For example, JP-A-57/101076 describes the partial or complete acetylation with acetic anhydride of amino-functionalized organopolysiloxanes having at least two amino groups at elevated temperatures of 100 to 110° C. EP-A-161 888 likewise describes completely acetylated amino-functionalized organopolysiloxanes—with two amino groups in the functionalized side chain. Here, too, the acetylation with acetic anhydride is carried out at relatively high temperatures (>68° C.)

The viscosities of the oils increase to a certain extent as a result of the complete acylation. When using organic anhydrides for example, acetic anhydride, as acylating agents, acids are formed, for example, acetic acid, which either protonate unreacted amino groups or else are present as the free acid. In both cases, and primarily at elevated temperature such as those described in the present examples, for organosiloxanes with reactive end groups (e.g. Si—OH or Si—O-alkyl), the condensation is catalyzed, which leads to a viscosity increase, which can sometimes no longer be controlled, and/or to gelation.

For applications in cosmetics, there is a need for oily organopolysiloxanes having completely acylated amino groups which simultaneously have hydrophilic chain ends.

The preparation of linear hydroxy-terminated organo-polysiloxanes having acylated amino groups with carboxylic anhydride in aqueous emulsion is described in DE-A-4211269. However, the amino groups are not completely acylated in this process.

### SUMMARY OF THE INVENTION

The invention provides hydroxy-terminated organopolysiloxanes (O) of the general formula (1),

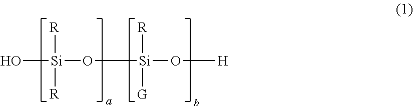

where
R is a monovalent unsubstituted or halogen-substituted hydrocarbon radical having 1 to 20 carbon atoms,
R¹ is a radical R, OR⁴ or —OH,
R⁴ is an alkyl radical having 1 to 6 carbon atoms,
G is a group of the general formula (2),

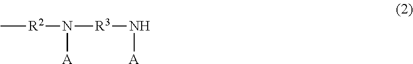

where
R², R³ are a divalent hydrocarbon radical having 1 to 6 carbon atoms, where nonadjacent —CH₂ units can be replaced by units which are selected from —C(═O)—, —O— and —S—,
A is R⁵—C(═O)—,
R⁵ is an alkyl radical having 1 to 20 carbon atoms,
a is an integer from 100 to 1500 and
b is an integer of at least 1.

The invention provides a process which surprisingly renders the viscosity increase of the hydroxy-terminated organopolysiloxanes (O) during the acylation step controllable and reproducible and thus permit the preparation of hydroxy-terminated, fully acylated organopolysiloxanes (O). Furthermore, the resulting organopolysiloxanes (O) are viscosity-stable.

The invention also provides aqueous emulsions (W) comprising 5% by weight to 70% by weight of the hydroxy-terminated organopolysiloxanes (O) of the general formula (1), and 1 to 150% by weight, based on the weight of the organopolysiloxane (O), of an emulsifier (E). The invention likewise provides the use of the emulsions (W) in compositions for cosmetics.

### DETAILED DESCRIPTION OF THE PREFERRED EMBODIMENTS

The emulsions (W) are characterized here by a very good storage stability and also dilution stability. The emulsions (W) can be used very readily in compositions for cosmetics such as shampoo or conditioner.

Areas of cosmetics in which the emulsions (W) are preferably used are hair cleansing and care, for example shampoos, conditioners, care rinses, hair masks, hair coloring products, styling products such as mousse and hair treatments such as permanent wave, and body washes, such as shower baths and soaps.

As a result of their content of organopolysiloxanes (O), the emulsions (W) for example bring about good care properties, good soft feel, increase in volume, anti-frizz, reduction in fly-away, reduction in combing force, heat protection and color protection in the hair.

The invention likewise provides a process A for preparing the hydroxy-terminated organopolysiloxanes (O), in which hydroxy-terminated organopolysiloxanes (N) of the general formula (1a)

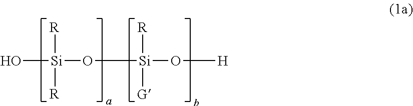

in which

G′ is a group of the general formula (2a)

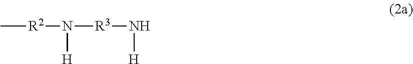

and R, R¹, R², R³, R⁴, a and b have the aforementioned meanings,

are reacted with acyclic carboxylic anhydrides of the general formula (3)

R⁵—C(═O)—O—C(═O)—R⁵ (3)

in a stoichiometrically equivalent amount to the amine groups present in the organopolysiloxane (N) in the presence of one or different emulsifiers (E1).

Surprisingly, by using the emulsifiers (E1), it is possible to control the viscosity of the reaction mixture very readily during the acylation.

After the reaction has taken place, the mixture of emulsifiers (E1) and organopolysiloxane (O) can be immediately emulsified so that the resulting oil viscosity of organopolysiloxane (O) remains stable. By virtue of the acylation to give organopolysiloxane (O) and its emulsification in one step, a very efficient process procedure is possible.

The invention likewise provides a process B for preparing the hydroxy-terminated organopolysiloxane (O) in which hydroxy-terminated organopolysiloxanes (N) of the general formula (1a)

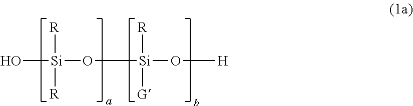

in which

G′ is a group of the general formula (2a)

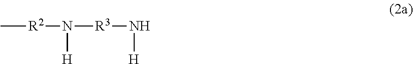

and R, R¹, R², R³, R⁴, a and b have the aforementioned meanings,

are reacted with acyclic carboxylic anhydrides of the general formula (3)

R⁵—C(═O)—O—C(═O)—R⁵ (3)

in a stoichiometrically equivalent amount to the amine groups present in the organopolysiloxane (N) without dilution, and after the acylation has taken place a neutralizing agent (B) is added immediately.

The resulting organopolysiloxane (O) can thus surprisingly be prepared in a viscosity-stable manner.

The procedure selected for both processes of using acyclic carboxylic anhydrides of the general formula (3) in a stoichiometrically equivalent amount to the amine groups present in organopolysiloxane (N) also has the advantage, in contrast to the excess of acetylating agent used in EP-A-161 888, that it is not necessary to distill off the excess at elevated temperatures, which in the case of the described hydroxy-terminated organopolysiloxanes, would inevitably lead to an increase in viscosity.

The conversion in a “stoichiometrically equivalent amount to the amine groups present in the organopolysiloxane (N)” means that per mole of nitrogen atom in the general formula (2a), 1 to at most 1.1, preferably at most 1.05 mol, in particular at most 1.01 mol, of acyclic carboxylic anhydride of the general formula (3) is used.

The monovalent hydrocarbon radical R can be halogen-substituted, linear, cyclic, branched, aromatic, saturated or unsaturated. Preferably, R has 1 to 6 carbon atoms, particular preference being given to linear alkyl radicals and phenyl radicals. Preferred halogen substituents are fluorine and chlorine. Particularly preferred monovalent hydrocarbon radicals R are methyl, ethyl, phenyl.

The alkyl radical R¹ is preferably selected from methyl, ethyl, n-propyl, isopropyl, hydroxy, methoxy, ethoxy, n-propoxy and isopropoxy.

Preferably, the radicals R², R³ are an ethylene, n-propylene, isobutylene or n-butylene radical. A particularly preferred radical G′ is the radical —(CH₂)₃NH(CH₂)NH₂.

R⁵ can be linear, cyclic or branched. Preferably, R⁵ has 1 to 6 carbon atoms, particularly preference being given to linear alkyl radicals, in particular methyl, ethyl, n-propyl or isopropyl.

a is preferably an integral value of at least 200, more preferably at least 300, and in particular at least 500, and at most 1300, more preferably at most 1100 and in particular at most 900.

b is preferably an integral value of at most 100, more preferably at most 10, and in particular at most 5.

Preferably, a and b are selected such that the organo-polysiloxane (O) has a viscosity of at least 100, more preferably at least 1000, yet more preferably at least 5000, and in particular at least 15,000 mPas and at most 500,000, more preferably at most 200,000, yet more preferably at most 100,000, and in particular at most 60,000 mPas.

The amine number of the organopolysiloxane (N) is preferably at least 0.001, in particular at least 0.01 mmol/g and at most 5, more preferably at most 1, yet more preferably at most 0.1, and in particular at most 0.05 mmol/g.

The amine number refers to the amount of moles of HCl which are required to neutralize 1 g of organopolysiloxane (N).

Emulsifiers (E) which can be used for preparing the aqueous emulsions (W) are all useful ionic and nonionic emulsifiers, either individually or as mixtures of different emulsifiers with which aqueous dispersions, in particular aqueous emulsions of organopolysiloxanes, are able to be prepared. Likewise, as is known, inorganic solids can be used as emulsifiers (E). These are e.g. silicas or bentonites as described in EP 1017745 A or DE 19742759 A.

Examples of anionic emulsifiers are:

1. Alkyl sulfates, particularly those with a chain length of 8 to 18 carbon atoms, alkyl and alkaryl ether sulfates with 8 to 18 carbon atoms in the hydrophobic radical and 1 to 40 ethylene oxide (EO) and/or propylene oxide (PO) units.

2. Sulfonates, particularly alkylsulfonates having 8 to 18 carbon atoms, alkylarylsulfonates having 8 to 18 carbon atoms, taurides, esters and half-esters of sulfosuccinic acid with monohydric alcohols or alkylphenols having 4 to 15 carbon atoms; optionally, these alcohols or alkylphenols can also be ethoxylated with 1 to 40 EO units.

3. Alkali metal and ammonium salts of carboxylic acids having 8 to 20 carbon atoms in the alkyl, aryl, alkaryl or aralkyl radical.

4. Phosphoric acid part esters and their alkali metal and ammonium salts, particularly alkyl and alkaryl phosphates having 8 to 20 carbon atoms in the organic radical, alkyl ether sulfates and alkaryl ether sulfates having 8 to 20 carbon atoms in the alkyl or alkaryl radical and 1 to 40 EO units.

Examples of nonionic emulsifiers are:

5. Polyvinyl alcohol which also has 5 to 50%, preferably 8 to 20 vinyl acetate units, with a degree of polymerization of 500 to 3000.

6. Alkyl polyglycol ethers, preferably those with 3 to 40 EO units and alkyl radicals of 8 to 20 carbon atoms.

7. Alkylaryl polyglycol ethers, preferably those with 5 to 40 EO units and 8 to 20 carbon atoms in the alkyl and aryl radicals.

8. Ethylene oxide/propylene oxide (EO/PO) block copolymers, preferably those with 8 to 40 EO and/or PO units.

9. Addition products of alkylamines with alkyl radicals from 8 to 22 carbon atoms with ethylene oxide or propylene oxide.

10. Fatty acids having 6 to 24 carbon atoms.

11. Alkyl polyglycosides of the general formula R*—O—ZO, in which R* is a linear or branched, saturated or unsaturated alkyl radical having on average 8-24 carbon atoms and ZO is an oligoglycoside radical having on average o=1-10 hexose or pentose units or mixtures thereof.

12. Natural substances and derivatives thereof, such as lecithin, lanolin, saponins, cellulose; cellulose alkyl ethers and carboxyalkylcelluloses, the alkyl groups of which each has up to 4 carbon atoms.

13. Linear organo(poly)siloxanes containing polar groups comprising in particular the elements O, N, C, S, P, Si, in particular those with alkoxy groups having up to 24 carbon atoms and/or up to 40 EO and/or PO groups.

Examples of cationic emulsifiers are:

14. Salts of primary, secondary and tertiary fatty amines having 8 to 24 carbon atoms with acetic acid, sulfuric acid, hydrochloric acid and phosphoric acids.

15. Quaternary alkyl- and alkylbenzeneammonium salts, in particular those whose alkyl groups have 6 to 24 carbon atoms, in particular the halides, sulfates, phosphates and acetates.

16. Alkylpyridinium, alkylimidazolinium and alkyl-oxazolinium salts, in particular those whose alkyl chain has up to 18 carbon atoms, specifically the halides, sulfates, phosphates and acetates.

Suitable ampholytic emulsifiers are particularly:

17. Long-chain substituted amino acids, such as N-alkyldi(aminoethyl)glycine or N-alkyl-2-aminopropionic acid salts.

18. Betaines, such as N-(3-acylamidopropyl)-N,N-dimethylammonium salts with a C₈-C₁₈-acyl radical and aklyamidazolium betaines.

Preferred emulsifiers are nonionic emulsifiers, in particular the alkyl polyglycol ethers listed above under 6., and cationic emulsifiers, in particular the quaternary alkyl- and alkylbenzeneammonium salts listed above under 15. The constituent (E) can consist of one of the aforementioned emulsifiers or of a mixture of two or more of the aforementioned emulsifiers, and it can be used in pure form or as solutions of one or more emulsifiers in water or organic solvents.

The invention further provides aqueous emulsions (W) comprising 5% by weight to 70% by weight of the hydroxy-terminated organopolysiloxanes (O) of the general formula (1), and 2 to 150% by weight, based on the weight of the organopolysiloxane (O), of an emulsifier (E).

The aqueous emulsions (W) consist of a discontinuous oil phase which comprises the acylated organopolysiloxane (1), the emulsifiers and the continuous water phase (oil-in-water emulsion).

The weight ratios of the discontinuous oil phase and of the continuous water phase can be varied within wide ranges. As a rule the fraction of the oil phase is 5 to 70% by weight, preferably 10 to 60% by weight, in each case based on the total weight of the emulsion (W). The fraction of emulsifiers is preferably in the range from 2 to 150% by weight, in particular up to 50% by weight, in each case based on the weight of the oil phase.

The preferred average particle size of the discontinuous oil phase is less than 2 μm, in particular less than 1 μm. Particular preference is given to average particle sizes of at most 0.8 μm, in particular at most 0.5 μm.

The emulsion (W) can comprise additives for certain purposes, for example for use in cosmetic compositions, alongside the above constituents. Suitable additives are for example biocides, such as fungicides, bactericides, algicides and microbicides, thickeners, antifreezes, antistats, dyes, fireproofing agents and organic plasticizers.

Emulsifiers (E1) which are used in process A during the acetylation must display essentially inert behavior towards the acyclic carboxylic anhydrides. Under this premise, they are preferably selected from the emulsifiers (E) described above.

Preferred emulsifiers (E1) are
(a) Alkyl polyglycol ethers, preferably those having 3 to 40 EO units and alkyl radicals of 8 to 20 carbon atoms.
(b) Alkylaryl polyglycol ethers, preferably those having 5 to 40 EO units and 8 to 20 carbon atoms in the alkyl and aryl radicals.
(c) Ethylene oxide/propylene oxide (EO/PO) block copolymers, preferably those having 8 to 40 EO and/or PO units.
(d) Addition products of alkylamines with alkyl radicals of 8 to 22 carbon atoms with ethylene oxide or propylene oxide.

or cationic emulsifiers such as
(e) Quaternary alkyl- and alkylbenzeneammonium salts, in particular those whose alkyl groups have 6 to 24 carbon atoms, in particular the halides, sulfates, phosphates and acetates.
(f) Alkylpyridinium, alkylimidazolinium and alkyloxazolinium salts, in particular those whose alkyl chain has up to 18 carbon atoms, specifically the halides, sulfates, phosphates and acetates.

Particularly preferred emulsifiers are nonionic emulsifiers, in particular those listed above under (a). The constituent (E1) can consist of one of the aforementioned emulsifiers or of a mixture of two or more of the aforementioned emulsifiers.

Neutralizing agents (B) which are used in preparation process B are alkaline acting salts such as alkali(ne earth) metal hydroxides, e.g. sodium hydroxide, potassium hydroxide, lithium hydroxide, alkali(ne earth) metal carbonates, e.g. potassium carbonate, sodium carbonate, lithium carbonate, alkali(ne earth) metal silanolates, e.g. sodium trimethylsilanolate, lithium trimethylsilanolate, alkali(ne earth) metal siloxanolates, e.g. lithium siloxanolate, alkali(ne earth) metal alkanolates, e.g. sodium methanolate, sodium ethanolate, potassium tert-butanolate or ammonium salts, e.g. ammonium hydroxide or organic amines, e.g. triethanolamine (TEA), diethanolamine, ethanolamine, triethylamine, and isopropylamine.

The pressures for processes A and B are preferably at least 0.010, in particular at least 0.05 MPa and preferably at most 10, more preferably at most 1 MPa. In particular, the pressure used is that which prevails at the production site.

The temperature in the case of process A is preferably at least 0° C., more preferably at least 10° C., and in particular at least 20° C. and preferably at most 100° C., more preferably at most 80° C., and in particular at most 60° C.

The temperature in the case of process B is preferably at least 0° C., more preferably at least 10° C., and in particular at least 20° C. and preferably at most 100° C., more preferably at most 80° C., and in particular at most 60° C.

The organopolysiloxane (1) can be obtained virtually emulsifier-free either by breaking the emulsion in any desired way, e.g. by adding water-soluble organic solvents, e.g. methanol, ethanol, isopropanol, acetone, or by adding salts, such as sodium chloride, or by extracting with organic solvents, e.g. n-hexane, n-heptane, mixtures of n-hexane/isopropanol, n-hexane/acetone.

All of the above symbols in the formulae above in each case have their meanings independently of one another. In all formulae, the silicon atom is tetravalent.

In the examples below, unless stated otherwise in each case, all quantitative data and percentages are based on the weight, all pressures are 0.10 MPa (abs.) and all temperatures are 20° C.

### EXAMPLE 1

Acetylation according to the invention by process B:

600 g of an amine oil of the general formula (1a) where R=methyl, R¹=methoxy, G′=3-((2-aminoethyl)amino)propyl and a and b are selected such that the oil has a viscosity of 14,100 mPas and an amine number of 0.032 mol/g is heated to 40° C. and admixed with 1.96 g (19.2 mmol; equimolar relative to the amine content) of acetic anhydride and then stirred for 30 min at 40° C. The reaction mixture is admixed with 2.86 g (19.2 mmol) of triethanolamine, stirred for 30 min at 40° C. and then cooled to room temperature. The acetylated amine oil of the general formula (1) has a viscosity of 22,200 mPas. After storage for 84 days at room temperature, the viscosity has increased to 29,800 Pas.

### EXAMPLE 1a

Acetylation not according to the invention:

600 g of an amine oil of the general formula (1a) where R=methyl, R¹=methoxy, G′=3-((2-aminoethyl)amino)propyl and a and b are selected such that the oil has a viscosity of 14,100 mPas and an amine number of 0.032 mol/g is heated to 40° C. and admixed with 1.96 g (19.2 mmol; equimolar relative to the amine content) of acetic anhydride and then stirred for 30 min at 40° C. After cooling to room temperature, the acetylated amine oil has a viscosity of 24,600 mPas. After storage for 84 days at room temperature, the viscosity has increased to 117,200 Pas.

Emulsion Preparation:

An emulsifying device (PC-Laborsystem) is charged with 5.26% by weight of isotridecyl hexaethoxylate, commercially available under the trade name Lutensol® IT6 (BASF), 3.45% by weight of an aqueous solution of cetyltrimethylammonium chloride (29% strength, available under the trade name Genamin® CTAC, Clariant) and 6.00% by weight of demineralized water. 55.00% by weight of organopolysiloxane of the general formula (1) are then added, and a preemulsion is prepared under high shear. This is diluted by the portionwise addition of in total 30.17% by weight of demineralized water. A pH of 3.5 is established by adding 0.02% by weight of acetic acid. This results in a milky-white stable emulsion which has an average particle size of 0.19 μm.

### EXAMPLE 2

Acetylation according to the invention by process A:

An emulsifying device (PC-Laborsystem) is charged with 5.26% by weight of isotridecyl hexaethoxylate, commercially available under the trade name Imbentin-T/060 (Kolb) and 55.00% by weight of an amine oil of the general formula (1a) where R=methyl, R¹=methoxy, G′=3-((2-aminoethyl)amino)propyl and a and b are selected such that the oil has a viscosity of 18 000 mPas and an amine number of 0.022 mol/g. 0.12% of acetic anhydride is added and the reaction mixture is stirred for 15 min. Then, 3.45% by weight of an aqueous solution of cetyltrimethylammonium chloride (29% strength, available under the trade name Genamin® CTAC, Clariant) and 6.00% by weight of demineralized water are added, and a preemulsion is prepared under high shear, and this is diluted by the portionwise addition of in total 30.17% by weight of demineralized water. This results in a milky-white stable emulsion which has an average particle size of 0.33 μm.

The desired organopolysiloxane of the general formula (1) is extracted from some of the emulsion with isopropanol/n-hexane. By means of ¹H-NMR, the virtually complete acetylation can be demonstrated by the two H₃C—CO signals at 1.87 ppm and 2.03 ppm.

### EXAMPLE 3

Acetylation according to the invention by process A:

The acetylation and emulsion preparation of example 2 is repeated in essence except that besides demineralized water the following starting materials were used: 3.45% by weight of isotridecyl hexaethoxylate, commercially available under the trade name Imbentin-T/060 (Kolb), 55.00% by weight of an amine oil of the general formula (1a) where R═R¹=methyl, G′=3-((2-aminoethyl)amino)propyl and a and b are selected such that the oil has a viscosity of 25,260 mPas and an amine number of 0.026 mol/g, 0.12% by weight of acetic anhydride, 3.45% by weight of an aqueous solution of cetyltrimethylammonium chloride (29% strength, available under the trade name Genamin® CTAC, Clariant). This results in a milky-white stable emulsion which has an average particle size of 0.26 μm.

The desired organopolysiloxane of the general formula (1) is extracted from some of the emulsion with isopropanol/n-hexane. By means of ¹H-NMR, the virtually complete acetylation can be demonstrated by the two H₃C—CO signals at 1.87 ppm and 2.03 ppm.

### EXAMPLE 4

Acetylation according to the invention by process A:

The acetylation and emulsion preparation of example 2 is repeated in essence except that besides demineralized water the following starting materials were used: 3.45% by weight of isotridecyl hexaethoxylate, commercially available under the trade name Imbentin-T/060 (Kolb), 55.00% by weight of an amine oil of the structure (1a) where R═R¹=methyl, G′=3-((2-aminoethyl)amino)propyl and a and b are selected such that the oil has a viscosity of 22,350 mPas and an amine number of 0.026 mol/g, 0.12% by weight of acetic anhydride, 3.45% by weight of an aqueous solution of cetyltrimethylammonium chloride (29% strength, available under the trade name Genamin® CTAC, Clariant). This results in a milky-white stable emulsion which has an average particle size of 0.33 μm.

The desired organopolysiloxane of the general formula (1) is extracted from some of the emulsion with isopropanol/n-hexane. It has a viscosity of 35,000 mPas (25° C.); this shows that as a result of the in-situ acetylation only a moderate and thus controllable increase in oil viscosity occurred. By means of ¹H-NMR, the virtually complete acetylation can be demonstrated by the two H₃C—CO signals at 1.87 ppm and 2.03 ppm.

### EXAMPLE 5

Use of the emulsion from example 4 in a shampoo or conditioner formulation:

Shampoo formulation

| INCI name | Trade name | Conc (%) |
|---|---|---|
| Aqua (water demin.) | Water | 4.49 |
| Guar Hydroxypropyltrimonium | N-Hance ® 3000 | 0.22 |
| Chloride | | |
| Sodium Laureth Sulfate | Genapol ® LRO 26.5% | 78.87 |
| Glycol Distearate | Genapol ® PMS | 1.32 |
| PEG-150 Distearate | Eumulgin ® EO 33 | 0.22 |
| Emulsion from example 4 | | 3.64 |
| Cocamidopropyl Betaine | Genagen ® CAB 818 | 11.07 |
| | 30% | |
| Methylchloroisothiazolinone | Kathon ® CG | 0.07 |
| and Methylisothiazolinone | | |
| Sodium Chloride | Sodium Chloride | 0.10 |
| | Solution (25%) | |

Conditioner formulation

| INCI name | Trade name | Conc (%) |
|---|---|---|
| Aqua (water demin.) | Water | 87.40 |
| Citric Acid | Citric Acid | 0.20 |
| Hydroxyethylcellulose | Tylose ® H 4000 P2 | 1.20 |
| Tetrasodium EDTA | EDTA ® B Powder | 0.20 |
| Polysorbate 80 | Tween ® 80 | 1.00 |
| Behentrimonium Chloride | Genamin ® KDMP | 1.76 |
| Cetyl Alcohol | Cetyl Alcohol | 1.00 |
| Stearamidopropyl | Incromine ® SD | 0.50 |
| Dimethylamine | | |
| Stearyl Alcohol | Stearyl Alcohol | 3.00 |
| Emulsion from example 4 | | 3.64 |
| Methylchloroisothiazolinone | Kathon ® CG | 0.10 |
| and Methylisothiazolinone | | |

1.-8. (canceled)

9. A hydroxy-terminated organopolysiloxane of the formula (1), 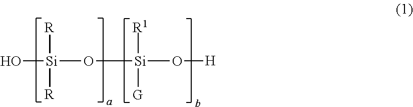
where
R is a monovalent unsubstituted or halogen-substituted hydrocarbon radical having 1 to 20 carbon atoms,
R¹ is a radical R, —OR⁴ or —OH,
R⁴ is an alkyl radical having 1 to 6 carbon atoms,
G is a group of the formula (2), 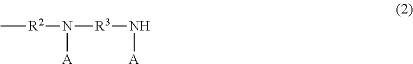
where
R², R³ are divalent hydrocarbon radicals having 1 to 6 carbon atoms, where nonadjacent —CH₂ units are optionally replaced by units —C(═O)—, —O— and —S—,
A is R⁵—C(═O)—,
R⁵ is an alkyl radical having 1 to 20 carbon atoms and
a is an integer from 100 to 1500 and
b is an integer of at least 1.

10. The organopolysiloxane of claim 9, in which R has 1 to 6 carbon atoms.

11. The organopolysiloxane of claim 9, in which the radicals R², R³ are individually ethylene, n-propylene, isobutylene or n-butylene.

12. The organopolysiloxane of claim 10, in which the radicals R², R³ are individually ethylene, n-propylene, isobutylene or n-butylene.

13. The organopolysiloxane of claim 9, in which R⁵ are linear alkyl radicals having 1 to 6 carbon atoms.

14. The organopolysiloxane of claim 10, in which R⁵ are linear alkyl radicals having 1 to 6 carbon atoms.

15. The organopolysiloxane of claim 11, in which R⁵ are linear alkyl radicals having 1 to 6 carbon atoms.

16. An aqueous emulsion comprising 5% by weight to 70% by weight of the hydroxy-terminated organopolysiloxanes of formula (1) of claims 9, and 1 to 150% by weight, based on the weight of the organopolysiloxane, of an emulsifier.

17. A cosmetic composition, comprising at least one emulsion of claim 16.

18. A process for preparing a hydroxy-terminated organopolysiloxane of claim 9, comprising reacting at least one hydroxy-terminated organopolysiloxane of the formula (1a) 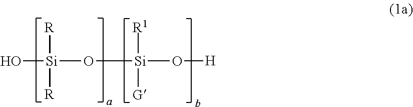
in which
G′ is a group of the formula (2a) 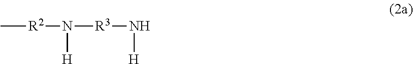
with acyclic carboxylic anhydride(s) of the formula (3)
R⁵—C(═O)—O—C(═O)—R⁵ (3)
in a stoichiometrically equivalent amount to the amine groups present in the organopolysiloxane, in the presence of one or more emulsifiers.

19. A process for preparing a hydroxy-terminated organopolysiloxane of claim 9, comprising reacting hydroxy-terminated organopolysiloxanes (N) of the general formula (1a) 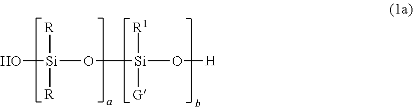
in which
G′ is a group of the formula (2a) 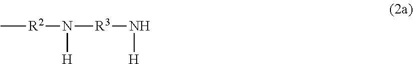
with acyclic carboxylic anhydrides of the formula (3)
R⁵—C(═O)—O—C(═O)—R⁵ (3)
in a stoichiometrically equivalent amount to the amine groups present in the organopolysiloxane without dilution, and after the acylation has taken place, promptly adding a neutralizing agent.
